(19) United States
(12) Patent Application Publication
(10) Pub. No.: US 2023/0274582 A1
(43) Pub. Date: Aug. 31, 2023

(54) DECEPTION DETECTION

(71) Applicants: Securiport LLC, Washington, DC (US); Department of Computer Science and Engineering University of Notre Dame, Notre Dame, IN (US)

(72) Inventors: Nathan Vance, Notre Dame, IN (US); Jeremy Speth, Notre Dame, IN (US); Siamul Karim Khan, Notre Dame, IN (US); Adam Czajka, Notre Dame, IN (US); Kevin W. Bowyer, Notre Dame, IN (US); Diane Wright, Notre Dame, IN (US); Patrick Flynn, Notre Dame, IN (US); Nathan Carpenter, Washington, DC (US); Leandro Olie, Washington, DC (US)

(21) Appl. No.: 18/115,414

(22) Filed: Feb. 28, 2023

Related U.S. Application Data
(60) Provisional application No. 63/314,589, filed on Feb. 28, 2022.

(51) Int. Cl. G06V 40/70; G06V 40/40; G06V 10/26; A61B 5/01; A61B 5/024; A61B 5/16

(52) U.S. Cl. CPC G06V 40/70; G06V 40/40; G06V 10/26; A61B 5/015; A61B 5/024; A61B 5/164; G06V 40/15

(57) ABSTRACT

Systems, devices, methods, and non-transitory computer-readable instructions for detecting deception of a subject from a media stream that capture a media stream of the subject including, the media stream including a sequence of frames, process each frame of the media stream to track a plurality of biometrics, and determine whether the subject in the media stream is deceptive based upon changes to respective biometrics.

### PRIORITY INFORMATION

This application claims the benefit of U.S. Provisional Patent Application No. 63/314,589 filed on Feb. 28, 2022, which is hereby incorporated by reference in its entirety.

### FIELD OF THE INVENTION

The embodiments of the present invention generally relate to use of biometrics, and more particularly, to media-based deception detection and remote physiological monitoring using a plurality of modalities, such as pulse.

### Discussion of the Related Art

In general, biometrics may be used to track vital signs that provide indicators about a subject's physical state that may be used in a variety of ways. As an example, for border security or health monitoring, vital signs may be used to screen for health risks (e.g., temperature). While sensing temperature is a well-developed technology, collecting other useful and accurate vital signs such as pulse rate (i.e., heart rate or heart beats per minute) or pulse waveform has required physical devices to be attached to the subject. The desire to perform biometric measurement without physical contact has produced some video-based techniques.

Performing reliable pulse rate or pulse waveform estimation from a camera sensor is more difficult than contact plethysmography for several reasons. The change in reflected light from the skin's surface, because of light absorption of blood, is very minor compared to those caused by changes in illumination. Even in settings with ambient lighting, the subject's movements drastically change the reflected light and overpower the pulse signal.

Currently, deception detection relies upon a variety of datasets, typically using a single sensing modality. Early researchers, inspired by the polygraph, believed information from the nervous system would likely give the best signals for deceit. Along these lines, the EEG-P300 dataset was developed, which consists solely of electroencephalography (EEG) data. Early researchers asserted that humans could be trained to detect deception with high accuracy, using micro-expressions. Inspired by human visual capabilities, other datasets contain high frame-rate RGB video, for more than 100 subjects. For example, the Box-of-Lies dataset was developed with RGB video and audio from a game show, and presents preliminary findings using linguistic, dialog, and visual features.

Multiple modalities have been introduced in the hope of enabling more robust deception detection. For example, the use of thermal imaging was introduced to a dataset for deception including RGB video, as well as physiological and audio recordings. Other researchers proposed the Bag-of-Lies, a multimodal dataset with gaze data for detecting deception in casual settings. Concerns about the authenticity of deception in constrained environments spurred the development of the Real-life Trial dataset. Transcripts and video from the courtroom were obtained from public multimedia sources to construct nearly an hour of authentic deception footage. While the environment for “high-stakes” behavior is more difficult to achieve in the lab setting, the number of free variables involved in retrospectively assembling a real-world dataset (e.g., camera resolution, angle, lighting, distance) makes algorithm design difficult.

Several other datasets have been developed. In the MAHNOB-HCI dataset, subjects' faces are relatively stationary (a significant limitation). PURE is the first dataset with stationary and moving faces. The MMSEHR dataset was used for rPPG during elicited emotion. To accommodate data requirements for deep learning-based solutions, the VIPL-HR dataset was developed. Aside from being the largest publicly available dataset for rPPG, preliminary results from a CNN outperformed then-existing techniques. Recently, the UBFC-RPPG dataset (containing rigid motion and a skin segmentation algorithm for rPPG) was released.

Although numerous datasets having been developed, remote (i.e., non-contact) high accuracy deception detection remains a challenge. Accordingly, the inventors have developed systems, devices, methods, and non-transitory computer-readable instructions that enable accurate deception detection without physical contact and with minimal constraints on the subject's movement and position.

### SUMMARY OF THE INVENTION

Accordingly, the present invention is directed to deception detection and remote physiological monitoring using a plurality of modalities that substantially obviates one or more problems due to limitations and disadvantages of the related art.

Additional features and advantages of the invention will be set forth in the description which follows, and in part will be apparent from the description, or may be learned by practice of the invention. The objectives and other advantages of the invention will be realized and attained by the structure particularly pointed out in the written description and claims hereof as well as the appended drawings.

To achieve these and other advantages and in accordance with the purpose of the present invention, as embodied and broadly described, the embodiments include systems, devices, methods, and non-transitory computer-readable instructions for detecting deception of a subject from a media stream that capture a media stream of the subject including, the media stream including a sequence of frames, process each frame of the media stream to track a plurality of biometrics, and determine whether the subject in the media stream is deceptive based upon changes to respective biometrics.

In connection with any of the various embodiments, the media stream includes one or more of a visible-light video stream, a near-infrared video stream, a longwave-infrared video stream, a thermal video stream, and an audio stream of the subject.

In connection with any of the various embodiments, the plurality of biometrics includes two or more of pulse rate, eye gaze, eye blink rate, pupil diameter, face temperature, speech, and micro-expressions.

In connection with any of the various embodiments, the plurality of biometrics includes pulse rate, pupil diameter, and face temperature.

In connection with any of the various embodiments, cropping each frame of the media stream to encapsulate a region of interest that includes one or more of a face, cheek, forehead, or an eye.

In connection with any of the various embodiments, the region of interest includes two or more body parts.

In connection with any of the various embodiments, combining at least two of a visible-light video stream, a near-infrared video stream, and a thermal video stream into a fused video stream.

In connection with any of the various embodiments, the visible-light video stream, the near-infrared video stream, and/or the thermal video stream are combined according to a synchronization device.

It is to be understood that both the foregoing general description and the following detailed description are examples and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

FIG. 1 illustrates a system for pulse waveform estimation.

FIG. 2 illustrates an analysis of images collected in different spectra.

FIG. 3 illustrates changes observed in a subject's pulse rate.

FIG. 4 illustrates a correlation between inferred and ground truth rPPG signals at each facial region.

FIG. 5 illustrates that a facial region can be divided into regions of interest.

FIG. 6 illustrates detection of circles fitting an iris and a pupil.

FIG. 7 illustrates a computer-implemented method for deception detection.

### DETAILED DESCRIPTION OF THE EXEMPLARY EMBODIMENTS

Reference will now be made in detail to the embodiments of the present invention, examples of which are illustrated in the accompanying drawings. Wherever possible, like reference numbers will be used for like elements.

Embodiments of user interfaces and associated methods for using a device are described. It should be understood, however, that the user interfaces and associated methods can be applied to numerous devices types, such as a portable communication device such as a tablet or mobile phone. The portable communication device can support a variety of applications, such as wired or wireless communications. The various applications that can be executed on the device can use at least one common physical user-interface device, such as a touchscreen. One or more functions of the touchscreen as well as corresponding information displayed on the device can be adjusted and/or varied from one application to another and/or within a respective application. In this way, a common physical architecture of the device can support a variety of applications with user interfaces that are intuitive and transparent.

The embodiments of the present invention provide systems, devices, methods, and non-transitory computer-readable instructions to measure one or more biometrics, including heart-rate and pulse waveform, without physical contact with the subject. In the various embodiments, the systems, devices, methods, and instructions collect, process, and analyze video taken in one or more modalities (e.g., visible light, near infrared, longwave infrared, thermal, pulse, gaze, blinking, pupillometry, face temperature, and micro-expressions, etc.) to detect deception from a distance without constraining the subject's movement or posture. New digital sensors expand the potential to address challenges in remote human monitoring.

For example, the pulse waveform for the subject's heartbeat may be used as a biometric input to establish features of the physical state of the subject and how they change over a period of observation (e.g., during questioning or other activity). Remote photoplethysmography (rPPG) is the monitoring of blood volume pulse from a camera at a distance. Using rPPG, blood volume pulse from video at a distance from the skin's surface may be detected. The disclosure of U.S. application Ser. No. 17/591,929, entitled “VIDEO BASED DETECTION OF PULSE WAVEFORM”, filed 3 Feb. 2022, is hereby incorporated by reference, in its entirety.

In various embodiments, changes to the subject's eye gaze, eye blink rate, pupil diameter, speech, face temperature, and micro-expressions are used to determine deception. EEG also can be used to predict anxiety and/or cognitive load. For example, pupil diameter distinguishes liars from truth tellers, finding that pupils dilate when a subject is attempting to mislead. In another example, eye movements, gestures, and posture also can be used to detect deceit.

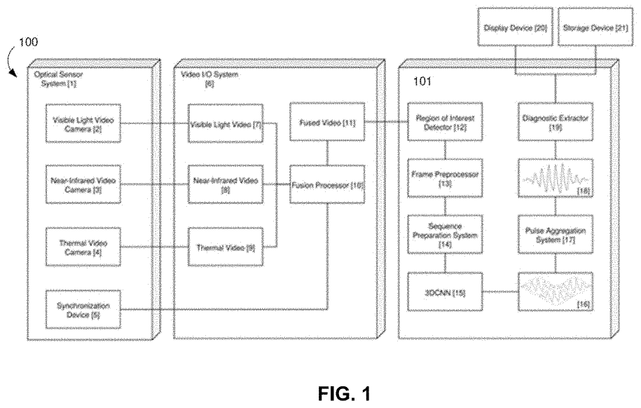

FIG. 1 illustrates a system 100 for pulse waveform estimation. System 100 includes optical sensor system 1, video I/O system 6, and video processing system 101.

Optical sensor system 1 includes one or more camera sensors, each respective camera sensor configured to capture a video stream including a sequence of frames. For example, optical sensor system 1 may include a visible-light camera 2, a near-infrared camera 3, a thermal camera 4, or any combination thereof. In the event that multiple camera sensors are utilized (e.g., single modality or multiple modality), the resulting multiple video streams may be synchronized according to synchronization device 5. Alternatively, or additionally, one or more video analysis techniques may be utilized to synchronize the video streams. Although a visible-light camera 2, a near-infrared camera 3, a thermal camera 4 are enumerated, other media devices can be used, such as a speech recorder.

Video I/O system 6 receives the captured one or more video streams. For example, video I/O system 6 is configured to receive raw visible-light video stream 7, near-infrared video stream 8, and thermal video stream 9 from optical sensor system 1. Here, the received video streams may be stored according to known digital format(s). In the event that multiple video streams are received (e.g., single modality or multiple modality), fusion processor 10 is configured to combine the received video streams. For example, fusion processor 10 may combine visible-light video stream 7, near-infrared video stream 8, and/or thermal video stream 9 into a fused video stream 11. Here, the respective streams may be synchronized according to the output (e.g., a clock signal) from synchronization device 5.

At video processing system 101, region of interest detector 12 detects (i.e., spatially locate) one or more spatial regions of interest (ROI) within each video frame. The ROI may be a face, another body part (e.g., a hand, an arm, a foot, a neck, etc.) or any combination of body parts. Initially, region of interest detector 12 determines one or more coarse spatial ROIs within each video frame. Region of interest detector 12 is robust to strong facial occlusions from face masks and other head garments. Subsequently, frame preprocessor 13 crops the frame to encapsulate the one or more ROI. In some embodiments, the cropping includes each frame being downsized by bi-cubic interpolation to reduce the number of image pixels to be processed. Alternatively, or additionally, the cropped frame may be further resized to a smaller image.

Sequence preparation system 14 aggregates batches of ordered sequences or subsequences of frames from frame processer 13 to be processed. Next, 3-Dimensional Convolutional Neural Network (3DCNN) 15 receives the sequence or subsequence of frames from the sequence preparation system 14. 3DCNN 15 processes the sequence or subsequence of frames, by a 3-dimensional convolutional neural network, to determine the spatial and temporal dimensions of each frame of the sequence or subsequence of frames and to produce a pulse waveform point for each frame of the sequence of frames. 3DCNN 15 applies a series of 3-dimensional convolution, averaging, pooling, and nonlinearities to produce a 1-dimensional signal approximating the pulse waveform 16 for the input sequence or subsequences.

In some configurations, pulse aggregation system 17 combines any number of pulse waveforms 16 from the sequences or subsequences of frames into an aggregated pulse waveform 18 to represent the entire video stream. Diagnostic extractor 19 is configured to compute the heart rate and the heart rate variability from the aggregated pulse waveform 18. To identify heart rate variability, the calculated heart rate of various subsequences may be compared. Display unit 20 receives real-time or near real-time updates from diagnostic extractor 19 and displays aggregated pulse waveform 18, heart rate, and heart rate variability to an operator. Storage Unit 21 is configured to store aggregated pulse waveform 18, heart rate, and heart rate variability associated with the subject.

Additionally, or alternatively, the sequence of frames may be partitioned into a partially overlapping subsequences within the sequence preparation system 14, wherein a first subsequence of frames overlaps with a second subsequence of frames. The overlap in frames between subsequences prevents edge effects. Here, pulse aggregation system 17 may apply a Hann function to each subsequence, and the overlapping subsequences added to generate aggregated pulse waveform 18 with the same number of samples as frames in the original video stream. In some configurations, each subsequence is individually passed to the 3DCNN 15, which performs a series of operations to produce a pulse waveform for each subsequence 16. Each pulse waveform output from the 3DCNN 15 is a time series with a real value for each video frame. Since each subsequence is processed by the 3DCNN 15 individually, they are subsequently recombined.

In some embodiments, one or more filters may be applied to the region of interest. For example, one or more wavelengths of LED light may be filtered out. The LED may be shone across the entire region of interest and surrounding surfaces or portions thereof. Additionally, or alternatively, temporal signals in non-skin regions may be further processed. For example, analyzing the eyebrows or the eye's sclera may identify changes strongly correlated with motion, but not necessarily correlated with the photoplethysmogram. If the same periodic signal predicted as the pulse is found on non-skin surfaces, it may indicate a non-real subject or attempted security breach.

Although illustrated as a single system, the functionality of system 100 may be implemented as a distributed system. While system 100 determines heart rate, other distributed configurations track changes to the subject's eye gaze, eye blink rate, pupil diameter, speech, face temperature, and micro-expressions, for example. Further, the functionality disclosed herein may be implemented on separate servers or devices that may be coupled together over a network, such as a security kiosk coupled to a backend server. Further, one or more components of system 100 may not be included. For example, system 100 may be a smartphone or tablet device that includes a processor, memory, and a display, but may not include one or more of the other components shown in FIG. 1. The embodiments may be implemented using a variety of processing and memory storage devices. For example, a CPU and/or GPU may be used in the processing system to decrease the runtime and calculate the pulse in near real-time. System 100 may be part of a larger system. Therefore, system 100 may include one or more additional functional modules.

According to the various embodiments, a deception detection and physiological monitoring (DDPM) dataset and baseline experiments with this dataset are described. DDPM data is collected in an interview context, in which the interviewee attempts to deceive the interviewer with selected responses. DDPM supports analysis of video and pulse data for facial features including pulse, gaze, eye movement, blink rate, pupillometry, face temperature, and micro-expressions, for example. The dataset comprises over eight (8) million high resolution RGB, near infrared (NIR), and thermal frames from face videos, along with cardiac pulse, blood oxygenation, audio, and deception-oriented interview data. The dataset is provided with evaluation protocols to accurately assess automated deception detection techniques.

The embodiments provide: (a) the largest deception detection dataset in terms of total truthful and deceptive responses, recording length, and raw data size; (b) the first dataset for both deception detection and remote pulse monitoring with RGB, near infrared, and thermal imaging modalities; (c) the first rPPG dataset with facial movement and expressions in a natural conversational setting; and (d) baseline results for deception detection using pupillometry, heart rate estimation, and feature fusion results. In addition, the embodiments include: (a) results from experiments probing the robustness of RPNet, a solution to rPPG; (b) a pupillometry method for working with low resolution video; (c) a feature fusion analysis utilizing rPPG, pupillometry, and thermal data for deception detection.

The DDPM dataset and initial baseline results are provided. For example, a context is an interview scenario in which the interviewee attempts to deceive the interviewer on selected responses. The interviewee is recorded in RGB, near infrared, and longwave infrared, along with cardiac pulse, blood oxygenation, and audio. After collection, data were annotated for interviewer/interviewee, curated, ground-truthed, and organized into train/test parts for a set of canonical deception detection experiments. Feature fusion experiments discovered that a combination of rPPG, pupil, and thermal data yielded the best deception detection results, with an equal error rate of 0.357. Subjects' heart rates were estimated from face videos (remotely) with a mean absolute error lower than 2 bpm. The database contains almost 13 hours of recordings of 70 subjects, and over eight (8) million visible-light, near-infrared, and thermal video frames, along with appropriate meta, audio, and pulse oximeter data. The DDPM dataset is the only dataset that includes recordings of five modalities in an interview scenario that can be used in both deception detection and remote photoplethysmography research as well as commercial applications.

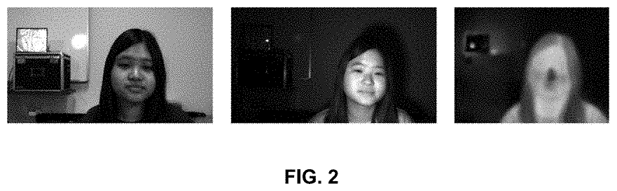

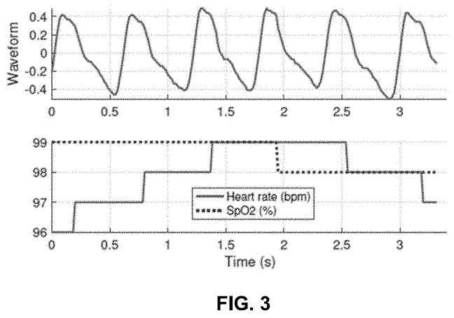

Detection of facial movements requires high spatial and temporal resolution. FIG. 2 illustrates the analyzing of images collected in different spectra. FIG. 2 shows sample images from the RGB, near infrared, and thermal cameras (left to right) from the collected DDPM dataset, and may provide deeper insight into facial cues associated with deception. Additionally, changes observed in the cardiac pulse rate as in FIG. 3 may elucidate a subject's emotional state. Speech dynamics such as tone changes provide another mode for detecting deception. An acquisition arrangement was assembled and composed of three cameras, a pulse oximeter, and a microphone.

For example, the sensing apparatus consisted of (i) a DFK 33UX290 RGB camera from The Imaging Source (TIS) operating at 90 FPS with a resolution of 1920×1080 px; (ii) A DMK 33UX290 monochrome camera from TIS with a bandpass filter to capture near-infrared images (730 to 1100 nm) at 90 FPS and 1920×1080 px; (iii) a FLIR C2 compact thermal camera that yielded 80×60 px images at 9 FPS; (iv) a FDA-certified Contec CMS50EA pulse oximeter that provides a 60 samples/second SpO2 and heart rate profile; and (v) a Jabra SPEAK 410 omni-directional microphone recording both interviewer and interviewee at 44.1 kHz with 16-bit audio measurements. The sensors were time-synchronized using visible and audible artifacts generated by an Arduino-controlled device. The media data were captured by a workstation designed to accommodate the continuous streaming of data from the three cameras (750 Mbps), operating a graphical user interface (GUI) that contained subject registration and interview progression components.

Deception metadata. Age, gender, ethnicity, and race were recorded for all participants. Each of the 70 interviews consisted of 24 responses, 9 of which were deceptive. Overall, 630 deceptive responses and 1050 honest responses were collected. To the inventors' knowledge, the 1,680 annotated responses is the most ever recorded in a deception detection dataset. The interviewee recorded whether they had answered as instructed for each question. For deceptive responses, they also rated how convincing they felt they were, on a 5-point Likert scale ranging from “I was not convincing at all” to “I was certainly convincing”. The interviewer recorded their belief about each response, on a 5-point scale from “certainly the answer was deceptive” to “certainly the answer was honest”. The data was additionally annotated to indicate which person (interviewer or interviewee) was speaking and the interval in time when they were speaking.

Data post-processing. The RGB and near infrared videos were losslessly compressed. The interviews' average, minimum, and maximum durations were 11 minutes, 8.9 minutes, and 19.9 minutes, respectively. In total, the DDPM dataset consists of 776 minutes of recording from each of the sensor modalities. The oximeter recorded SpO2, heart rate, and pulse waveform at 60 Hz giving average heart rates for the whole interview ranging from 40 bpm to 161 bpm.

Pulse Detection Experiments. Five pulse detection techniques were evaluated on the DDPM dataset, relying on blind-source separation, chrominance, and color space transformations, and deep learning.

Methods. The general pipeline for pulse detection contains region selection, spatial averaging, a transformation or signal decomposition, and frequency analysis. For region selection, OpenFace was used to detect 68 facial landmarks used to define a face bounding box (e.g., region of interest). The bounding box was extended horizontally by 5% on each side, and by 30% above and 5% below, and then converted to a square with a side length that was the larger of the expanded horizontal and vertical sizes, to ensure that the cheeks, forehead and jaw were contained. For the chrominance-based approaches, the skin pixels within the face were utilized.

Given the region of interest, a channel-wise spatial averaging was used to produce a 1D temporal signal for each channel. The blind source separation approaches apply independent component analysis (ICA) to the channels, while the chrominance-based approaches combine the channels to define a robust pulse signal. The heart rate is then found over a time window by converting the signal to the frequency domain and selecting the peak frequency fp as the cardiac pulse. The heart rate is computed as dHR=60×fp beats per minute (bpm).

For training the deep learning-based approach, RPNet was used, a 3D Convolutional Neural Network (3DCNN) that is fed with the face cropped at the bounding box and downsized to 64×64 pixels with bicubic interpolation. During training and evaluation, the model is given clips of the video consisting of 136 frames (i.e., 1.5 seconds). Here, 136 frames was used as the the minimum time for an entire heartbeat to occur, considering 40 bpm as a lower bound for average subjects. RPNet was configured to minimize the negative Pearson correlation between predicted and normalized ground truth pulse waveforms.

The oximeter recorded ground truth waveform and heart rate estimates at 60 Hz and upsampled to 90 Hz to match the RGB camera frame rate. One of the difficulties in defining an oximeter waveform as a target arises from the phase difference observed at the face and finger, coupled with time lags from the acquisition apparatus. To mitigate the phase shift, the output waveform predicted by CHROM (chosen because it does not require supervised training) was used to shift the ground truth waveform such that the cross-correlation between them is maximized. The ground truth waveforms contain infrequent noisy segments caused by subjects moving their fingers inside the pulse oximeter. These segments are detected as jumps in heart rate over 7 bpm in a second, as calculated using a FFT with bandpass bounds of 40 and 160 bpm and a sliding window of 10 seconds. If such a jump occurs, that 10 second FFT window is marked as invalid and masked from the dataset.

The Adam optimizer was used with a learning rate of α=0.0001, and parameter values of β1=0.99 and β2=0.999 to train the model for 50 epochs, then select the model with the lowest loss on the validation set as the final model.

For videos longer than the clip length of 136 frames, it is necessary to perform predictions in sliding window fashion over the full video. A stride of half the clip length was used to slide across the video. The windowed outputs are standardized, a Hann function is applied to mitigate edge effects from convolution, and they are added together to produce a single value per frame.

Pulse detection performance is analyzed by calculating the error between predicted and ground truth heart rates. The heart rate is calculated by applying a 10 second wide Hamming window to the signal and converting to the frequency domain, from which the index of the maximum spectral peak between 0.66 Hz and 3 Hz (40 bpm to 180 bpm) is selected as the heart rate. Since the frequency domain suffers quantization effects, spectral peaks were dequantized by taking the weighted average of spectral readings between adjacent valleys. Metrics from the rPPG literature were used to evaluate performance, such as mean absolute error (MAE), root mean squared error (RMSE), and Pearson correlation coefficient for the pulse waveform, r wave. It was found that while masking out noisy sections from the ground truth improved evaluation metrics for CHROM and RPNet, it degraded results for the other methods. As such, masking was applied only to CHROM and RPNet.

The original blind-source separation approach, POH10, is outperformed by POH11 due to signal detrending and filtering, which removes noise from motion. Both chrominance-based approaches perform similarly, although POS gives good accuracy without filtering. RPNet was evaluated using 5-fold cross validation, reporting the mean and 95% confidence interval for each performance metric. RPNet outperforms the non-deep learning baselines.

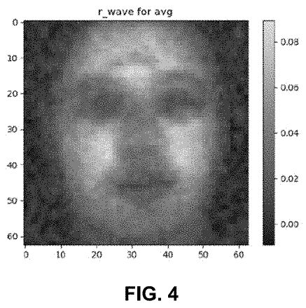

In the various embodiments, the inventors identified which regions of the face produce the best rPPG results. FIG. 4 illustrates the correlation between inferred and ground truth rPPG signals at each facial region. The cheeks and forehead give a rPPG signal that is more correlated with the ground truth than other parts of the face. The heatmap of FIG. 4 was generated by performing an evaluation using (for each subject) a 2×2 pixel region from every location across the 64×64 pixel video. These 632 regions were then averaged across subjects, and each region corresponds to a single pixel in the heatmap. From the image, it is understood the cheeks and forehead produce a better rPPG wave than other facial skin, which is plausible since those regions are more highly vascularized than other parts of the face.

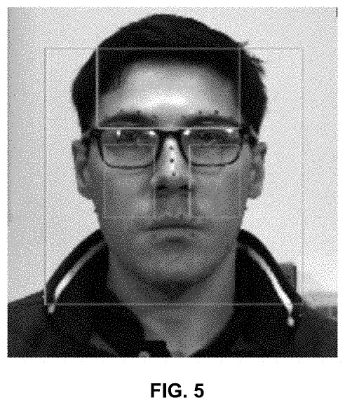

In the various embodiments, RPNet performance is improved by focusing it on regions with a stronger signal, i.e., the forehead and cheeks. The facial region can be divided into the three regions or regions of interest (e.g., forehead, right cheek, left cheek) as shown in FIG. 5. Using the models trained over the full face, an rPPG was inferred wave over these regions. The forehead obtained the most accurate results of the subregions, although even when the three regions are combined, RPNet utilizing the full frame still outperforms these more focused regions.

Landmarker Study. As discussed above, the OpenFace landmarker was used in the image processing pipeline for the purpose of generating bounding boxes because it exhibits superior landmark stability, resulting in a low amount of jitter in the landmarks. However, alternatives, such as MediaPipe can be used, especially due to its applicability in real-time systems. MediaPipe performs nearly as well as OpenFace as a bounding box method for rPPG, despite its poorer bounding box stability (as measured by average displacement in pixels between successive frames), exhibiting an increase in MAE by only 33% despite a degradation in bounding box stability by 260%. As MediaPipe is easier to implement, its uses is expected for real-time rPPG systems.

Pupil Size Estimation. The general pipeline for pupil detection contains eye region selection, and estimation of the pupil and iris radii. For selecting the eye region, OpenFace was utilized to detect 68 facial landmarks (e.g., utilizing the same detections as in Pulse detection) and utilized the points around the eyelid to define an eye bounding box. The bounding box is configured to have a 4:3 aspect ratio by lengthening the shorter side (which is usually the vertical side).

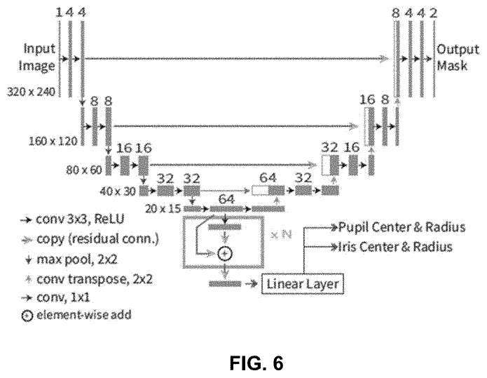

To detect the pupil and iris radii, a modified CC-Net architecture is used. In particular, the encodings from the CC-Net are used to configure a CNN regressor to detect circles fitting the iris and pupil as illustrated in FIG. 6. For the pupil and iris circle parameters, boundary points were traced for the pupil and iris in the masks and fit circles into these points using RANSAC. Then, the modified CC-Net architecture was configured to predict both the mask, and the pupil and iris circle parameters. To evaluate the performance of the model on the DDPM dataset, eye regions were extracted randomly, ensuring that the eye is open, from the DDPM dataset and manually annotate circles for pupil and iris. Different architectures can be used for the CNN regressor. It was observed that residual connections improve the results as deeper networks were used.

Fusion. On or more modalities can be used for deception detection including pulse, gaze, eye movement (e.g., Saccadic), blink rate, pupillometry, face temperature, and micro-expressions, for example. The combination of rPPG, pupillometry, and thermal data is effective for deception detection. As a standalone feature, rPPG is effective. The feature fusion using of these three features obtains an equal error rate of 0.357, exceeding any of these features individually.

The Deception Detection and Physiological Monitoring (DDPM) dataset is described, the most comprehensive dataset to date in terms of number of different modalities and volume of raw video, to support exploration of deception detection and remote physiological monitoring in a natural conversation setup. The sensors are temporally synchronized, and imaging across visible, near infrared and longwave infrared spectra provides more than 8 million high-resolution images from almost 13 hours of recordings in a deception-focused interview scenario. Along with this dataset, baseline results are provided for heart rate detection, and the feasibility of deception detection using pupillometry, heart rate, and thermal data.

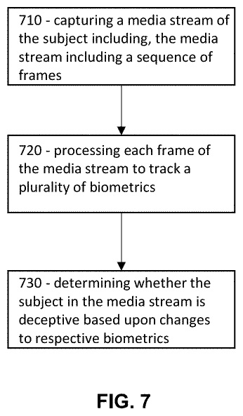

FIG. 7 illustrates a computer-implemented method for deception detection.

At 710, the method captures a media stream of the subject including, the media stream including a sequence of frames. The video stream may include one or more of a visible-light video stream, a near-infrared video stream, and a thermal video stream of a subject. In some instances, the method can combine at least two of the visible-light video stream, the near-infrared video stream, and/or the thermal video stream into a fused video stream to be processed. The visible-light video stream, the near-infrared video stream, and/or the thermal video stream are combined according to a synchronization device and/or one or more video analysis techniques.

Next, at 720, the method processes each frame of the media stream to track changes in a plurality of biometrics. For example, the plurality of biometrics include two or more of pulse rate, eye gaze, eye blink rate, pupil diameter, face temperature, speech, and micro-expressions.

Lastly, the method determines whether the subject in the media stream is deceptive based upon changes to respective biometrics. For example, changes to the subject's eye gaze, eye blink rate, pupil diameter, speech, face temperature, and micro-expressions are used to determine deception, at 730/

It will be apparent to those skilled in the art that various modifications and variations can be made in the deception detection and remote physiological monitoring using a plurality of modalities of the present invention without departing from the spirit or scope of the invention. Thus, it is intended that the present invention cover the modifications and variations of this invention provided they come within the scope of the appended claims and their equivalents.

What is claimed is:

1. A computer-implemented method for detecting deception of a subject from a media stream, the computer-implemented method comprising:
capturing a media stream of the subject including, the media stream including a sequence of frames;
processing each frame of the media stream to track a plurality of biometrics; and
determining whether the subject in the media stream is deceptive based upon changes to respective biometrics.

2. The computer-implemented method according to claim 1, wherein the media stream includes one or more of a visible-light video stream, a near-infrared video stream, a longwave-infrared video stream, a thermal video stream, and an audio stream of the subject.

3. The computer-implemented method according to claim 1, wherein the plurality of biometrics includes two or more of pulse rate, eye gaze, eye blink rate, pupil diameter, face temperature, speech, and micro-expressions.

4. The computer-implemented method according to claim 1, wherein the plurality of biometrics includes pulse rate, pupil diameter, and face temperature.

5. The computer-implemented method according to claim 1, further comprising cropping each frame of the media stream to encapsulate a region of interest that includes one or more of a face, cheek, forehead, or an eye.

6. The computer-implemented method according to claim 5, wherein the region of interest includes two or more body parts.

7. The computer-implemented method according to claim 1, further comprising:
combining at least two of a visible-light video stream, a near-infrared video stream, and a thermal video stream into a fused video stream.

8. The computer-implemented method according to claim 7, wherein the visible-light video stream, the near-infrared video stream, and/or the thermal video stream are combined according to a synchronization device.

9. A system for detecting deception of a subject from a media stream, the system comprising:
a processor; and
a memory storing one or more programs for execution by the processor, the one or more programs including instructions for:
capturing a media stream of the subject, the media stream including a sequence of frames;
processing each frame of the media stream to track a plurality of biometrics; and
determining whether the subject in the media stream is deceptive based upon changes to respective biometrics.

10. The system according to claim 9, wherein the media stream includes one or more of a visible-light video stream, a near-infrared video stream, a longwave-infrared video stream, a thermal video stream, and an audio stream of the subject.

11. The system according to claim 9, wherein the plurality of biometrics includes two or more of pulse rate, eye gaze, eye blink rate, pupil diameter, face temperature, speech, and micro-expressions.

12. The system according to claim 9, wherein the plurality of biometrics includes pulse rate, pupil diameter, and face temperature.

13. The system according to claim 9, further comprising cropping each frame of the media stream to encapsulate a region of interest that includes one or more of a face, cheek, forehead, or an eye.

14. The system according to claim 13, wherein the region of interest includes two or more body parts.

15. The system according to claim 9, further comprising:
combining at least two of a visible-light video stream, a near-infrared video stream, and a thermal video stream into a fused video stream.

16. The system according to claim 15, wherein the visible-light video stream, the near-infrared video stream, and/or the thermal video stream are combined according to a synchronization device.
